(19) United States
(12) Patent Application Publication
(10) Pub. No.: US 2009/0155767 A1
(43) Pub. Date: Jun. 18, 2009

(54) Methods for a predictive diagnostic test for tamoxifen

(76) Inventors: David L. Rimm, Branford, CT (US); Jennifer M. Giltnane, Nashville, TN (US); Lisa Ryden, Angelholm (SE)

Correspondence Address: COOPER & DUNHAM, LLP, 30 Rockefeller Plaza, 20th Floor, NEW YORK, NY, 10112, US

(21) Appl. No.: 12/215,458

(22) Filed: Jun. 27, 2008

Related U.S. Application Data
(60) Provisional application No. 60/937,698, filed on Jun. 29, 2007.

(51) Int. Cl. C12Q 1/00

(52) U.S. Cl. 435/4

(57) ABSTRACT

A method for determining the likelihood that a therapy involving administration of tamoxifen to a patient afflicted with an estrogen receptor positive breast cancer will provide a therapeutic benefit to the patient which comprises determining the level of expression of epidermal growth factor receptor present within a non-nuclear compartment in cells present in a breast tissue sample from the patient; and comparing the level of expression so obtained to a predetermined level of expression wherein the likelihood the therapy will provide a therapeutic benefit to the patient is greater if the level of expression in step a) is less than the predetermined level of expression.

This application claims the benefit of U.S. Provisional Application No. 60/937,698, filed Jun. 29, 2007, the entire contents of which is hereby incorporated by reference into this application.

Throughout this application, various publications are referenced by Arabic numerals within parentheses. Full citations for these publications may be found at the end of the specification immediately preceding the claims. The disclosures of each of these publications is hereby incorporated by reference into this application in order to more fully describe the state of the art as known to those skilled therein as of the date of this application.

1. Field of the Invention

This invention relates to the field of quantitative measurement of epidermal growth factor receptor as a negative predictor for tamoxifen response in hormone receptor positive premenopausal breast cancer.

2. Background of the Invention

Treatment with anti-estrogen therapies such as tamoxifen has substantially decreased the risk of recurrence and mortality in women with hormone receptor-positive disease (1). Unfortunately, both de novo and acquired resistance remain a major clinical problem and the mechanisms for resistance are under active investigation (2-6). Possible causes for tumor resistance include loss of estrogen receptors, tamoxifen-stimulated tumor growth, variant receptor or receptor-interacting protein expression, and cross-talk with growth factor signaling pathways (7). The interaction with growth factor pathways seems very promising based on studies that show biological evidence of functional cross-talk (8).

The human epidermal growth factor receptor (EGFR) is a transmembrane tyrosine kinase receptor whose activation sustains programs of cell proliferation, survival, and migration (9). Establishing the incidence and prognostic significance of EGFR expression in breast cancer has been problematic due to differences in antibody specificity, inter-laboratory irreproducibility, and the presence of EGFR isoforms. However, a trend has clearly been established that EGFR overexpression, while infrequent in breast cancer, is associated with reduced survival and resistance to endocrine therapies (2-5, 10, 11). In fact, in vitro studies have shown that estrogen deprivation induces activation of parallel growth and survival pathways, including EGFR expression, while pre- and post-treatment tumor samples from patients demonstrating tamoxifen resistance show increased EGFR ligand and receptor expression (12).

Historically, EGFR has been a difficult protein to assess. Even using well validated antibodies, there is very little quantitative in situ data on EGFR expression. Automated quantitative analysis (AQUA®) of protein expression is a novel, immunofluorescence-based quantitative method of measuring protein expression in situ (13). See for example U.S. Pat. No. 7,219,016, which is a description of the AQUA method and the entire contents of which is hereby incorporated by reference into this application. This technique has been previously validated in breast cancer, and AQUA scores are directly correlated with in situ protein concentration as measured by ELISA (14). The technology is now commercially available (HistoRx, New Haven Conn.) and has been published in over 40 peer reviewed papers from 9 labs. In this study, we employed AQUA to measure EGFR expression in tumor samples from a randomized clinical trial of tamoxifen in premenopausal, early-stage breast cancer patients to assess the affect of EGFR protein levels on recurrence-free survival (RFS) in each arm of the trial.

### SUMMARY OF THE INVENTION

This invention provides a method for determining the likelihood that a therapy involving administration of tamoxifen to a patient afflicted with a breast cancer will provide a therapeutic benefit to the patient which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein there is a likelihood the therapy will provide the therapeutic benefit to the patient if the level of expression determined in step a) is less than the predetermined reference level of expression.

This invention provides a method for identifying a patient afflicted with a breast cancer who will likely obtain a therapeutic benefit from tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the patient will likely obtain the therapeutic benefit from tamoxifen therapy if the level of expression determined in step a) is less than the predetermined reference level of expression.

This invention provides a method for determining whether a breast cancer will be responsive to tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the breast cancer will be responsive to tamoxifen if the level of expression determined in step a) is less than the predetermined reference level of expression.

This invention provides a method for determining whether a breast cancer will not be responsive to tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the breast cancer will be not responsive to tamoxifen therapy if the level of expression determined in step a) is greater than the predetermined reference level of expression.

This invention provides a method for determining the likelihood that a therapy involving administration of an aromatase inhibitor to a patient afflicted with a breast cancer will provide a therapeutic benefit to the patient which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy wherein there is a likelihood the therapy will provide the therapeutic benefit to the patient if the level of expression determined in step a) is less than the predetermined reference level of expression.

### BRIEF DESCRIPTION OF THE FIGURES

FIG. 1: Immunofluorescent Immunohistochemistry for Automated Quantitative Analysis (AQUA)

(A-D) Tumor histospot with EGFR-AQUA score of 74.94 in non-nuclear compartment

(E-H) Tumor histospot with EGFR-AQUA score of 3.14 in non-nuclear compartment

(A and E) Cytokeratin-Cy2 image (10×), used to identify tumor within each histospot

(B and F) Pseudo-colored colocalization image demonstrating compartment assignment (20×): Cytokeratin membrane coalescence (Green) was used to define the non-nulcear compartment , DAPI staining (Blue) defined the nuclear compartment.

(C and G) Binary gating of cytokeratin expression created the tumor mask (White) for AQUA measurements (20×); histospots with <5% tumor per core were excluded from analysis

(D and H) Post-RESA image of EGFR-Cy5 image (20×); measurements for EGFR expression were from the non-nuclear compartment in all histospots; inset in (H) with brightness/contrast increased to show background signal.

FIG. 2: Distribution and Reproducibility of AQUA Scores

(A) AQUA-EGFR score distribution for 16 cancer cell line controls, with scores ranging from 5.60-178.06. (B) AQUA scores were normalized by log-normal transformation for linear regression analysis. Agreement between two independent samples from each patient was high (R=0.865). (C) IHC scores for EGFR expression, scored by Dako-PharmDx guidelines, ranged from 0-3 in 412 evaluable patient samples. 56 patients (13.6%) had IHC scores>0. (D) Average AQUA scores for EGFR expression ranged from 1.33 to 72.52 in 523 evaluable samples from 327 unique patients. (E) Distribution of AQUA scores by IHC results. Scores by the two assays were moderately correlated (Spearman Rho=0.426, p<0.0001). (F) AQUA scores for HER2 expression ranged from 1.14 to 132.34 in 377 evaluable patient samples. (G) Correlation between AQUA-EGFR and AQUA-HER2 was not observed in this cohort (Spearman Rho=0.035, p=0.5662).

FIG. 3: Survival Analysis by EGFR Expression

(A) Frequency distribution of AQUA scores for ER+ patients, showing cutpoint of 4.909, at median for entire cohort. (B) Kaplan-Meier recurrence-free survival analysis by treatment (tamoxifen vs. control) of ER+ patients with low AQUA-EGFR scores (p=0.01), and (C) ER+ patients with high AQUA-EGFR scores (p=0.69)

### DETAILED DESCRIPTION OF THE INVENTION

This invention provides a method for determining the likelihood that a therapy involving administration of tamoxifen to a patient afflicted with a breast cancer will provide a therapeutic benefit to the patient which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein there is a likelihood the therapy will provide the therapeutic benefit to the patient if the level of expression determined in step a) is less than the predetermined reference level of expression.

As used in this application, the term “level of expression” means either the level of mRNA that encodes estrogen growth factor receptor or the level of estrogen growth factor receptor protein.

As used in this application, a tumor tissue sample can refer to various kinds of tissue samples, for example, a tissue section.

As used in the method of this invention, the predetermined reference level is preferably 60 pg/ml or less, more preferably 40 pg/ml or less, still more preferably 20 pg/ml or less, yet more preferably 10 pg/ml or less, and most preferably 5 pg/ml or less.

The level of expression of mRNA that encodes estrogen growth factor receptor may be determined by using, for example, a Northern blot, a Reverse Northern blot, real-time polymerase chain reaction, or reverse transcription polymerase chain reaction.

The level of expression of epidermal growth factor receptor may be determined by measuring the level of epidermal growth factor receptor protein expression, for example, by using an ELISA procedure, an immunohistochemistry procedure, a quantitative image analysis procedure, a Western Blot, mass spectrometry, a quantitative immunohistochemistry procedure, or an automated pathology system.

The currently preferred methods of determining the level of expression involve use of immunohistochemistry procedures and quantitative image analysis. Numerous immunohistochemistry procedures for determining a level of expression are known in the art.

The level of expression may also be determined using an immunobased assay. The immunobased assays may use various antibodies or binding reagents with sensitivity. Examples of these antibodies include EGFR PharmDx (Dako) and 31G7 (invitrogen).

The level of expression may also be determined using an histological based assay. For example, by laser microdissection followed by mass spectrometry or by AQUA® analysis.

The level of epidermal growth factor receptor protein expression may be determined by a quantitative image analysis procedure.

Numerous quantitative image analysis procedures for determining a level of expression are known in the art. Examples of quantitative image analysis procedures that may be used to determine the level of expression include AQUA® procedures, as described in issued U.S. Pat. No. 7,219,016, which is incorporated by reference into this application in its entirety, the Bliss system, the ACIS system, the IVision and GenoMx system, the ScanScope Systems, the Ariol SL-50 System, and the LSC system which are available from the following respective manufacturers: Bacus Laboratories, Inc., Clarient, Inc., BioGenex, DakoCytomation, Applied Imaging Corporation, and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers. The disclosures of these publications is hereby incorporated by reference into this application.

The level of epidermal growth factor receptor protein expression may be determined in a cellular compartment within the tissue, or a subcellular compartment within the tissue.

The level of epidermal growth factor receptor protein expression may be determined in a non-nuclear compartment.

The level of expression may be determined in a cellular membrane compartment.

The method of this invention may be used to determine the level of expression of epidermal growth factor receptor present in various subcellular compartments, including, but not limited to, the non-nuclear compartment and the cellular membrane compartment. For example, other subcellular compartments may include the endoplasmic reticulum, golgi apparatus, or lysosome.

The breast cancer may be hormone receptor positive breast cancer.

The preceding description and method relates to breast cancer. This method may also be applicable to other types of cancers in which estrogen growth factor receptor expression correlates with a likelihood of benefit of tamoxifen or other pharmaceutical agents.

While the method of this invention may be particularly useful for use in assessing patients who have breast cancer, specifically estrogen receptor positive breast cancer, the method of this invention may also be used in connection with other forms of cancer, for example, colon cancer or colorectal cancer.

The patient may be a premenopausal female patient.

The level of expression of epidermal growth factor receptor may be determined using an automated pathology system.

The therapeutic benefit may be progression free or recurrence free survival.

As used herein, a “therapeutic benefit” includes but is not limited to recurrence free survival or progression free survival of the patient. Other therapeutic benefits include delay of recurrence of disease in patients where there is recurrence.

This invention provides a method for identifying a patient afflicted with a breast cancer who will likely obtain a therapeutic benefit from tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the patient will likely obtain the therapeutic benefit from tamoxifen therapy if the level of expression determined in step a) is less than the predetermined reference level of expression.

As used in this application, the term “level of expression” means either the level of mRNA that encodes estrogen growth factor receptor or the level of estrogen growth factor receptor protein.

As used in this application, a tumor tissue sample can refer to various kinds of tissue samples, for example, a tissue section.

As used in the method of this invention, the predetermined reference level is preferably 60 pg/ml or less, more preferably 40 pg/ml or less, still more preferably 20 pg/ml or less, yet more preferably 10 pg/ml or less, and most preferably 5 pg/ml or less.

The level of expression of mRNA that encodes estrogen growth factor receptor may be determined by using, for example, a Northern blot, a Reverse Northern blot, real-time polymerase chain reaction, or reverse transcription polymerase chain reaction.

The level of expression of epidermal growth factor receptor may be determined by measuring the level of epidermal growth factor receptor protein expression, for example, by using an ELISA procedure, an immunohistochemistry procedure, a quantitative image analysis procedure, a Western Blot, mass spectrometry, a quantitative immunohistochemistry procedure, or an automated pathology system.

The currently preferred methods of determining the level of expression involve use of immunohistochemistry procedures and quantitative image analysis. Numerous immunohistochemistry procedures for determining a level of expression are known in the art.

The level of expression may also be determined using an immunobased assay. The immunobased assays may use various antibodies or binding reagents with sensitivity. Examples of these antibodies include EGFR PharmDx (Dako) and 31G7 (invitrogen).

The level of expression may also be determined using an histological based assay. For example, by laser microdissection followed by mass spectrometry or by AQUA® analysis.

The level of epidermal growth factor receptor protein expression may be determined by a quantitative image analysis procedure.

Numerous quantitative image analysis procedures for determining a level of expression are known in the art. Examples of quantitative image analysis procedures that may be used to determine the level of expression include AQUA® procedures, as described in issued U.S. Pat. No. 7,219,016, which is incorporated by reference into this application in its entirety, the Bliss system, the ACIS system, the IVision and GenoMx system, the ScanScope Systems, the Ariol SL-50 System, and the LSC system which are available from the following respective manufacturers: Bacus Laboratories, Inc., Clarient, Inc., BioGenex, DakoCytomation, Applied Imaging Corporation, and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers. The disclosures of these publications is hereby incorporated by reference into this application.

The level of epidermal growth factor receptor protein expression may be determined in a cellular compartment within the tissue, or a subcellular compartment within the tissue.

The level of epidermal growth factor receptor protein expression may be determined in a non-nuclear compartment.

The level of expression may be determined in a cellular membrane compartment.

The method of this invention may be used to determine the level of expression of epidermal growth factor receptor present in various subcellular compartments, including, but not limited to, the non-nuclear compartment and the cellular membrane compartment. For example, other subcellular compartments may include the endoplasmic reticulum, golgi apparatus, or lysosome.

The breast cancer may be hormone receptor positive breast cancer.

The preceding description and method relates to breast cancer. This method may also be applicable to other types of cancers in which estrogen growth factor receptor expression correlates with a likelihood of benefit of tamoxifen or other pharmaceutical agents.

While the method of this invention may be particularly useful for use in assessing patients who have breast cancer, specifically estrogen receptor positive breast cancer, the method of this invention may also be used in connection with other forms of cancer, for example, colon cancer or colorectal cancer.

The patient may be a premenopausal female patient.

The level of expression of epidermal growth factor receptor may be determined using an automated pathology system.

The therapeutic benefit may be progression free or recurrence free survival.

As used herein, a “therapeutic benefit” includes but is not limited to recurrence free survival or progression free survival of the patient. Other therapeutic benefits include delay of recurrence of disease in patients where there is recurrence.

This invention provides a method for determining whether a breast cancer will be responsive to tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the breast cancer will be responsive to tamoxifen if the level of expression determined in step a) is less than the predetermined reference level of expression.

As used in this application, the term “level of expression” means either the level of mRNA that encodes estrogen growth factor receptor or the level of estrogen growth factor receptor protein.

As used in this application, a tumor tissue sample can refer to various kinds of tissue samples, for example, a tissue section.

As used in the method of this invention, the predetermined reference level is preferably 60 pg/ml or less, more preferably 40 pg/ml or less, still more preferably 20 pg/ml or less, yet more preferably 10 pg/ml or less, and most preferably 5 pg/ml or less.

The level of expression of mRNA that encodes estrogen growth factor receptor may be determined by using, for example, a Northern blot, a Reverse Northern blot, real-time polymerase chain reaction, or reverse transcription polymerase chain reaction.

The level of expression of epidermal growth factor receptor may be determined by measuring the level of epidermal growth factor receptor protein expression, for example, by using an ELISA procedure, an immunohistochemistry procedure, a quantitative image analysis procedure, a Western Blot, mass spectrometry, a quantitative immunohistochemistry procedure, or an automated pathology system.

The currently preferred methods of determining the level of expression involve use of immunohistochemistry procedures and quantitative image analysis. Numerous immunohistochemistry procedures for determining a level of expression are known in the art.

The level of expression may also be determined using an immunobased assay. The immunobased assays may use various antibodies or binding reagents with sensitivity. Examples of these antibodies include EGFR PharmDx (Dako) and 31G7 (invitrogen).

The level of expression may also be determined using an histological based assay. For example, by laser microdissection followed by mass spectrometry or by AQUA® analysis.

The level of epidermal growth factor receptor protein expression may be determined by a quantitative image analysis procedure.

Numerous quantitative image analysis procedures for determining a level of expression are known in the art. Examples of quantitative image analysis procedures that may be used to determine the level of expression include AQUA® procedures, as described in issued U.S. Pat. No. 7,219,016, which is incorporated by reference into this application in its entirety, the Bliss system, the ACIS system, the IVision and GenoMx system, the ScanScope Systems, the Ariol SL-50 System, and the LSC system which are available from the following respective manufacturers: Bacus Laboratories, Inc., Clarient, Inc., BioGenex, DakoCytomation, Applied Imaging Corporation, and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers. The disclosures of these publications is hereby incorporated by reference into this application.

The level of epidermal growth factor receptor protein expression may be determined in a cellular compartment within the tissue, or a subcellular compartment within the tissue.

The level of epidermal growth factor receptor protein expression may be determined in a non-nuclear compartment.

The level of expression may be determined in a cellular membrane compartment.

The method of this invention may be used to determine the level of expression of epidermal growth factor receptor present in various subcellular compartments, including, but not limited to, the non-nuclear compartment and the cellular membrane compartment. For example, other subcellular compartments may include the endoplasmic reticulum, golgi apparatus, or lysosome.

The breast cancer may be hormone receptor positive breast cancer.

The preceding description and method relates to breast cancer. This method may also be applicable to other types of cancers in which estrogen growth factor receptor expression correlates with a likelihood of benefit of tamoxifen or other pharmaceutical agents.

While the method of this invention may be particularly useful for use in assessing patients who have breast cancer, specifically estrogen receptor positive breast cancer, the method of this invention may also be used in connection with other forms of cancer, for example, colon cancer or colorectal cancer.

The patient may be a premenopausal female patient.

The level of expression of epidermal growth factor receptor may be determined using an automated pathology system.

The therapeutic benefit may be progression free or recurrence free survival.

As used herein, a “therapeutic benefit” includes but is not limited to recurrence free survival or progression free survival of the patient. Other therapeutic benefits include delay of recurrence of disease in patients where there is recurrence.

This invention provides a method for determining whether a breast cancer will not be responsive to tamoxifen therapy which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy; wherein the breast cancer will be not responsive to tamoxifen therapy if the level of expression determined in step a) is greater than the predetermined reference level of expression.

As used in this application, the term “level of expression” means either the level of mRNA that encodes estrogen growth factor receptor or the level of estrogen growth factor receptor protein.

As used in this application, a tumor tissue sample can refer to various kinds of tissue samples, for example, a tissue section.

As used in the method of this invention, the predetermined reference level is preferably 60 pg/ml or less, more preferably 40 pg/ml or less, still more preferably 20 pg/ml or less, yet more preferably 10 pg/ml or less, and most preferably 5 pg/ml or less.

The level of expression of mRNA that encodes estrogen growth factor receptor may be determined by using, for example, a Northern blot, a Reverse Northern blot, real-time polymerase chain reaction, or reverse transcription polymerase chain reaction.

The level of expression of epidermal growth factor receptor may be determined by measuring the level of epidermal growth factor receptor protein expression, for example, by using an ELISA procedure, an immunohistochemistry procedure, a quantitative image analysis procedure, a Western Blot, mass spectrometry, a quantitative immunohistochemistry procedure, or an automated pathology system.

The currently preferred methods of determining the level of expression involve use of immunohistochemistry procedures and quantitative image analysis. Numerous immunohistochemistry procedures for determining a level of expression are known in the art.

The level of expression may also be determined using an immunobased assay. The immunobased assays may use various antibodies or binding reagents with sensitivity. Examples of these antibodies include EGFR PharmDx (Dako) and 31G7 (invitrogen).

The level of expression may also be determined using an histological based assay. For example, by laser microdissection followed by mass spectrometry or by AQUA® analysis.

The level of epidermal growth factor receptor protein expression may be determined by a quantitative image analysis procedure.

Numerous quantitative image analysis procedures for determining a level of expression are known in the art. Examples of quantitative image analysis procedures that may be used to determine the level of expression include AQUA® procedures, as described in issued U.S. Pat. No. 7,219,016, which is incorporated by reference into this application in its entirety, the Bliss system, the ACIS system, the IVision and GenoMx system, the ScanScope Systems, the Ariol SL-50 System, and the LSC system which are available from the following respective manufacturers: Bacus Laboratories, Inc., Clarient, Inc., BioGenex, DakoCytomation, Applied Imaging Corporation, and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers. The disclosures of these publications is hereby incorporated by reference into this application.

The level of epidermal growth factor receptor protein expression may be determined in a cellular compartment within the tissue, or a subcellular compartment within the tissue.

The level of epidermal growth factor receptor protein expression may be determined in a non-nuclear compartment.

The level of expression may be determined in a cellular membrane compartment.

The method of this invention may be used to determine the level of expression of epidermal growth factor receptor present in various subcellular compartments, including, but not limited to, the non-nuclear compartment and the cellular membrane compartment. For example, other subcellular compartments may include the endoplasmic reticulum, golgi apparatus, or lysosome.

The breast cancer may be hormone receptor positive breast cancer.

The preceding description and method relates to breast cancer. This method may also be applicable to other types of cancers in which estrogen growth factor receptor expression correlates with a likelihood of benefit of tamoxifen or other pharmaceutical agents.

While the method of this invention may be particularly useful for use in assessing patients who have breast cancer, specifically estrogen receptor positive breast cancer, the method of this invention may also be used in connection with other forms of cancer, for example, colon cancer or colorectal cancer.

The patient may be a premenopausal female patient.

The level of expression of epidermal growth factor receptor may be determined using an automated pathology system.

The therapeutic benefit may be progression free or recurrence free survival.

As used herein, a “therapeutic benefit” includes but is not limited to recurrence free survival or progression free survival of the patient. Other therapeutic benefits include delay of recurrence of disease in patients where there is recurrence.

This invention provides a method for determining the likelihood that a therapy involving administration of an aromatase inhibitor to a patient afflicted with a breast cancer will provide a therapeutic benefit to the patient which comprises determining the level of expression of epidermal growth factor receptor in a breast tissue sample from the patient; and comparing the level of expression so obtained to a predetermined reference level of expression of epidermal growth factor receptor in breast cancer tissue samples of patients who had a lack of therapeutic benefit from such a therapy wherein there is a likelihood the therapy will provide the therapeutic benefit to the patient if the level of expression determined in step a) is less than the predetermined reference level of expression.

As used in this application, the term “level of expression” means either the level of mRNA that encodes estrogen growth factor receptor or the level of estrogen growth factor receptor protein.

As used in this application, a tumor tissue sample can refer to various kinds of tissue samples, for example, a tissue section.

As used in the method of this invention, the predetermined reference level is preferably 60 pg/ml or less, more preferably 40 pg/ml or less, still more preferably 20 pg/ml or less, yet more preferably 10 pg/ml or less, and most preferably 5 pg/ml or less.

The level of expression of mRNA that encodes estrogen growth factor receptor may be determined by using, for example, a Northern blot, a Reverse Northern blot, real-time polymerase chain reaction, or reverse transcription polymerase chain reaction.

The level of expression of epidermal growth factor receptor may be determined by measuring the level of epidermal growth factor receptor protein expression, for example, by using an ELISA procedure, an immunohistochemistry procedure, a quantitative image analysis procedure, a Western Blot, mass spectrometry, a quantitative immunohistochemistry procedure, or an automated pathology system.

The currently preferred methods of determining the level of expression involve use of immunohistochemistry procedures and quantitative image analysis. Numerous immunohistochemistry procedures for determining a level of expression are known in the art.

The level of expression may also be determined using an immunobased assay. The immunobased assays may use various antibodies or binding reagents with sensitivity. Examples of these antibodies include EGFR PharmDx (Dako) and 31G7 (invitrogen).

The level of expression may also be determined using an histological based assay. For example, by laser microdissection followed by mass spectrometry or by AQUA® analysis.

The level of epidermal growth factor receptor protein expression may be determined by a quantitative image analysis procedure.

Numerous quantitative image analysis procedures for determining a level of expression are known in the art. Examples of quantitative image analysis procedures that may be used to determine the level of expression include AQUA® procedures, as described in issued U.S. Pat. No. 7,219,016, which is incorporated by reference into this application in its entirety, the Bliss system, the ACIS system, the IVision and GenoMx system, the ScanScope Systems, the Ariol SL-50 System, and the LSC system which are available from the following respective manufacturers: Bacus Laboratories, Inc., Clarient, Inc., BioGenex, DakoCytomation, Applied Imaging Corporation, and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers. The disclosures of these publications is hereby incorporated by reference into this application.

The level of epidermal growth factor receptor protein expression may be determined in a cellular compartment within the tissue, or a subcellular compartment within the tissue.

The level of epidermal growth factor receptor protein expression may be determined in a non-nuclear compartment.

The level of expression may be determined in a cellular membrane compartment.

The method of this invention may be used to determine the level of expression of epidermal growth factor receptor present in various subcellular compartments, including, but not limited to, the non-nuclear compartment and the cellular membrane compartment. For example, other subcellular compartments may include the endoplasmic reticulum, golgi apparatus, or lysosome.

The breast cancer may be hormone receptor positive breast cancer.

The preceding description and method relates to breast cancer. This method may also be applicable to other types of cancers in which estrogen growth factor receptor expression correlates with a likelihood of benefit of tamoxifen or other pharmaceutical agents.

While the method of this invention may be particularly useful for use in assessing patients who have breast cancer, specifically estrogen receptor positive breast cancer, the method of this invention may also be used in connection with other forms of cancer, for example, colon cancer or colorectal cancer.

The patient may be a premenopausal female patient.

The level of expression of epidermal growth factor receptor may be determined using an automated pathology system.

The therapeutic benefit may be progression free or recurrence free survival.

As used herein, a “therapeutic benefit” includes but is not limited to recurrence free survival or progression free survival of the patient. Other therapeutic benefits include delay of recurrence of disease in patients where there is recurrence.

The aromatase inhibitor may be anastrozole (arimidex®).

The aromatase inhibitor may be exemestane (aromasin®).

The aromatase inhibitor that may be letrozole(femara®).

Tamoxifen may also be used to treat infertility in women with anovulatory disorders. A dose of 10-40 mg per day is administered in days 3-7 of a woman's cycle. In addition, a rare condition occasionally treated with tamoxifen is retroperitoneal fibrosis.

In men, tamoxifen may sometimes be used to treat gynecomastia that arises for example as a side effect of antiandrogen prostate cancer treatment. Tamoxifen may also be used by bodybuilders to prevent or reduce drug-induced gynecomastia caused by the estrogenic metabolites of anabolic steroids. Tamoxifen may also be used to treat or prevent gynecomastia in sex offenders undergoing treatment by temporary chemical castration.

Tamoxifen may also be effective in the treatment of mania in patients with bipolar disorder by blocking protein kinase C (PKC), an enzyme that regulates neuron activity in the brain. Researchers believe PKC is over-active during the mania in bipolar patients.

The following Experimental Details are set forth to aid in an understanding of the subject matter of this disclosure, but are not intended to, and should not be construed to, limit in any way the claims which follow thereafter.

### Experimental Details

### Synopsis

Purpose: Although there is evidence for interaction between Epidermal Growth Factor Receptor (EGFR) and Estrogen Receptor (ER), it is still not clear how this affects response to endocrine therapies like Tamoxifen. Here the relationship between EGFR expression and Tamoxifen response is assessed with a new quantitative technology.

Patients and Methods: A tissue microarray was constructed from breast cancer from a cohort of 564 patients enrolled in a randomized clinical trial for adjuvant tamoxifen treatment in early breast cancer with median follow-up of 14 years. EGFR expression was measured using AQUA™, a fluorescence-based method for quantitative analysis of in situ protein expression.

Results: In ER+ cases, Tamoxifen treated patients with low EGFR expression (n=113), showed a significant effect by two years of adjuvant tamoxifen (p=0.01), contrasting to no treatment effect in the EGFR high group (n=73, p=0.69). The untreated group showed 49% vs 57% 10-year RFS for EGFR low vs high (p=0.466) in the corresponding group of ER+ patients. A significant beneficial effect of tamoxifen treatment was seen in the EGFR low group; HR 0.43 (95% CI: 0.22-0.84), p=0.013 in contrast to no effect in the EGFR high group; HR 1.14 (95% CI: 0.59-2.22) p=0.7 and by using a Cox model.

Conclusion: This study provides clinical evidence that confirms the basic work that has shown high EGFR can indicate resistance to Tamoxifen. It suggests that careful measurement of EGFR protein expression might define a subset of low stage patients that could benefit from an alternative therapy.

### Patients and Methods

### Patients

From 1986 to 1991, 564 patients enrolled in a a randomized clinical trial for adjuvant tamoxifen treatment in early breast cancer (Swedish SBII:2a)(15). Criteria for enrollment included premenopausal status or age less than 50 years, with stage II invasive breast cancer. Patients were randomized to receive two years of tamoxifen (n=276) or no treatment (control, n=288), and were followed for disease-free and overall survival, with a median follow-up of 13.9 years. A detailed description of the trial and results has previously been published (15,16). The two study groups were equivalent in almost all analyzed tumor and clinical characteristics (Table 1).

### Tissue Microarray (TMA) Construction

Tumor specimens were analyzed in a tissue microarray format detailed in a previous publication (16) where specimens from 500 patients out of the 564 enrolled in the trial were available as formalin-fixed, paraffin-embedded tissue blocks. Representative areas of invasive cancer were selected from each block, and two 0.6 mm cores from each tumor block were arrayed in a recipient block.

### Cell Lines

A TMA containing cores from formalin-fixed, paraffin embedded cell pellets was used as a control for staining and AQUA analysis. A431, SK-BR-3, BT-474, MDA-MB-453, BT-549, T-47D, SW-480, MCF-7, MDA-MB-468, and MDA-MB-231 cell lines were purchased from the American Type Culture Collection (Manassas, Va.). BAF3 cells were obtained from a laboratory in the Department of Genetics at Yale University, and JEG-3, SKOV3, and EGFR-transfected CHO cells were obtained from the Maihle laboratory at Yale University (17). Culture conditions and cell line TMA construction have been published in detail elsewhere (14, 18). Our laboratory protocol for processing cell lines is also available on the web (http://www.tissuearray.org).

### Immunohistochemistry (IHC)

Slides were stained by a modified indirect immunofluorescence method as described previously (13). The arrays were deparaffinized with xylene and alcohol and rehydrated in water. Dako EGFR pharmDx kit (Dako Corporation, Carpinteria, Calif.) was used to detect EGFR expression. Package insert instructions were strictly followed with minor modifications to allow for quantitative AQUA analysis with immunofluorescent visualization. Specifically, after Proteinase K enzymatic digestion and EGFR primary antibody incubation, slides were incubated with a wide-spectrum screening rabbit anti-cow cytokeratin antibody (Dako Z0622) at 1:100 for 60 minutes at room temperature. This was followed by a 60 minute incubation with Alexa 546-conjugated goat anti-rabbit (A11010, Molecular Probes, Eugene, Oreg.) diluted 1:100 in the supplied Envision reagent. Cy5 directly conjugated to tyramide at a 1:50 dilution (SAT-705A, Perkin-Elmer, Boston, Mass.) was substituted for the DAB+ chromagen supplied in the kit. Prolong Gold mounting medium (P36931, Molecular Probes) containing 4′,6-Diamidino-2-phenylindole (DAPI) was used to define tissue nuclei.

Staining of tissue microarrays for HER-2/neu for AQUA analysis has been previously described (19). In this study, rabbit polyclonal anti-erbB-2 antibody A0485 (Dako) was used at 1:8000. Positive and negative controls were included in a specialized “boutique” array stained simultaneously containing 40 cases from a previously-described breast carcinoma tissue microarray (19) as well as 27 cell lines exhibiting variable levels of expression for each marker analyzed (see FIG. 2).

### Automated Quantification of Protein Expression (AQUAS™)

Complete and detailed descriptions image collection and of the AQUA™ method for analysis have been published previously (13, 20). Briefly, a binary image (tumor mask) was created from the cytokeratin image of each histospot, representing areas of epithelium. Histospots were excluded if the tumor mask represented less than 5% of the total histospot area. DAPI immunoreactivity defined the nuclear compartment. The non-nuclear compartment was defined by the tumor mask with specific exclusion of the nuclear compartment. Target expression was quantified by calculating Cy5 fluorescent signal intensity on a scale of 0-255 within each image pixel. An AQUA score was generated by dividing the sum of target signals within the tumor mask by the area of the membrane compartment. After validation of images to ensure adequate tumor sampling and absence of normal epithelium, the scores from two non-overlapping images were averaged for each patient case.

### Patient Classification

For analysis of tamoxifen response, only the ER+ subset of patients were included (16). By IHC, a cut-off value of at least 10% nuclear immunoreactivity was used to classify patients as ER-positive. Classification of EGFR expression was arbitrarily established by using the median value of the entire trial cohort (AQUA score=4.909) as a cut-off point for continuous AQUA values. In comparison, Chinese Hamster Ovary (CHO) cells, which do not express any detectable EGFR, had a background AQUA score of 5.60, thus any EGFR expression above background was considered positive.

### Statistical Methods

The statistical calculations were performed using SPSS Version 13.0 (SPSS, Chicago, Ill.) and Stata Version 9.2 (StataCorp, College Station, Tex.). Pearson's correlation coefficient (R) was used to assess the correlation between log AQUA-EGFR scores from redundant tumor cores and Spearman's Rho to assess the correlation between AQUA-EGFR score and other variables. Recurrence-free survival (RFS) was chosen as endpoint in the present study. RFS included breast cancer specific death, distant, regional and local recurrences and all analyses were performed with the intention to treat rule. The AQUA-EGFR score was dichotomized at the median in order to avoid bias problems associated with cut-off optimization. Kaplan-Meier plots were used to illustrate the survival in high and low EGFR, respectively, and the log rank test to test for equality of survival curves. Hazard ratios were estimated using Cox regression. Proportional hazards assumptions were checked using Schoenfeld's test. The null hypothesis of equal treatment effects in high and low EGFR were evaluated using a Cox model with a term for the interaction between EGFR class and treatment. Cox models with continuous AQUA-EGFR score were also fitted. The original score and a logarithmic transformation of the score were evaluated. The model fit was approximately the same for the two alternative models so the linear score was chosen. All p-values corresponded to two-sided tests and values less than 0.05 were considered significant.

### Results

### Cohort

Tumor specimens were available for 500 of the initially 564 included patients (Table 1). Most patients (84%) were less than 50 years of age at the time of diagnosis, and all patients had stage II invasive breast cancer. Although almost 1 in 4 patients demonstrated some lymph node metastasis, only 9/564 (<2%) received adjuvant chemotherapy (15). Tumor size was less than 2 cm in 37% of cases, and 62% of patients were classified as having ER+ breast cancer. This observation is in line with findings in other studies of premenopausal breast cancer cohorts, which tend to have a smaller percentage of hormone receptor positive disease (21). Since tamoxifen resistance may be influenced by failure to rigorously exclude ER− patients, we limited survival analysis to 324 patients with ER+ breast cancer, as previously defined by immunohistochemistry (15).

TABLE 1

| | Original study | | | Biomarker study | | |
|---|---|---|---|---|---|---|
| | Control arm | Tamoxifen arm | All patients* | Control | Tam | All |
| Variable | (n = 288) | (n = 276) | (n = 564) | n = 255 | n = 245 | n = 500 |
| Age | | | | | | |
| <40 | 61 | 52 | 113 | 50 | 43 | 93 |
| 40-49 | 184 | 178 | 362 | 168 | 161 | 329 |
| 50+ | 43 | 46 | 89 | 37 | 41 | 78 |
| Lymph node | | | | | | |
| status | | | | | | |
| 0 | 77 | 83 | 160 | 67 | 72 | 139 |
| 1-3 | 140 | 135 | 275 | 125 | 123 | 248 |
| 4+ | 70 | 57 | 127 | 62 | 49 | 111 |
| Unknown | 1 | 1 | 2 | 1 | 1 | 2 |
| Tumor size (mm) | | | | | | |
| 0-20 | 122 | 85 | 207 | 107 | 77 | 184 |
| 21+ | 166 | 190 | 356 | 148 | 167 | 315 |
| Unknown | 0 | 1 | 1 | 0 | 1 | 1 |
| ER Status | | | | | | |
| ER− | 104 | 102 | 206 | 72 | 79 | 151 |
| ER+ | 175 | 157 | 332 | 173 | 151 | 324 |
| Unknown | 9 | 17 | 26 | 10 | 15 | 25 |
| ER+/EGFR-AQUA | | | | | | |
| <Median (Low) | | | | 67 | 46 | 113 |
| >Median (High) | | | | 38 | 35 | 73 |
| Not interpretable | | | | 68 | 70 | 138 |

*A more detailed cohort description has been published previously (including tumor specimen collection) (15)

### Immunofluorescent Staining of EGFR in Breast Cancer Tissue Microarrays.

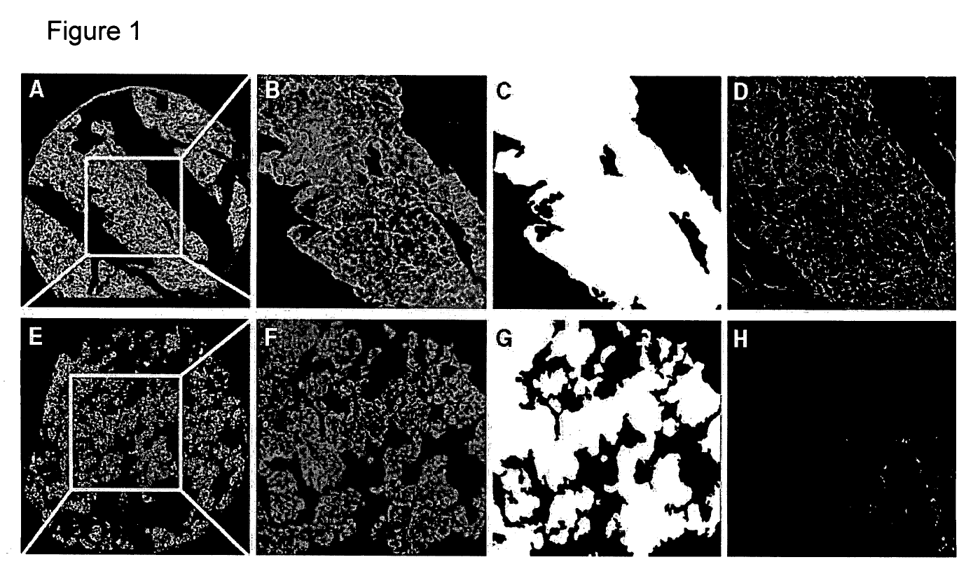

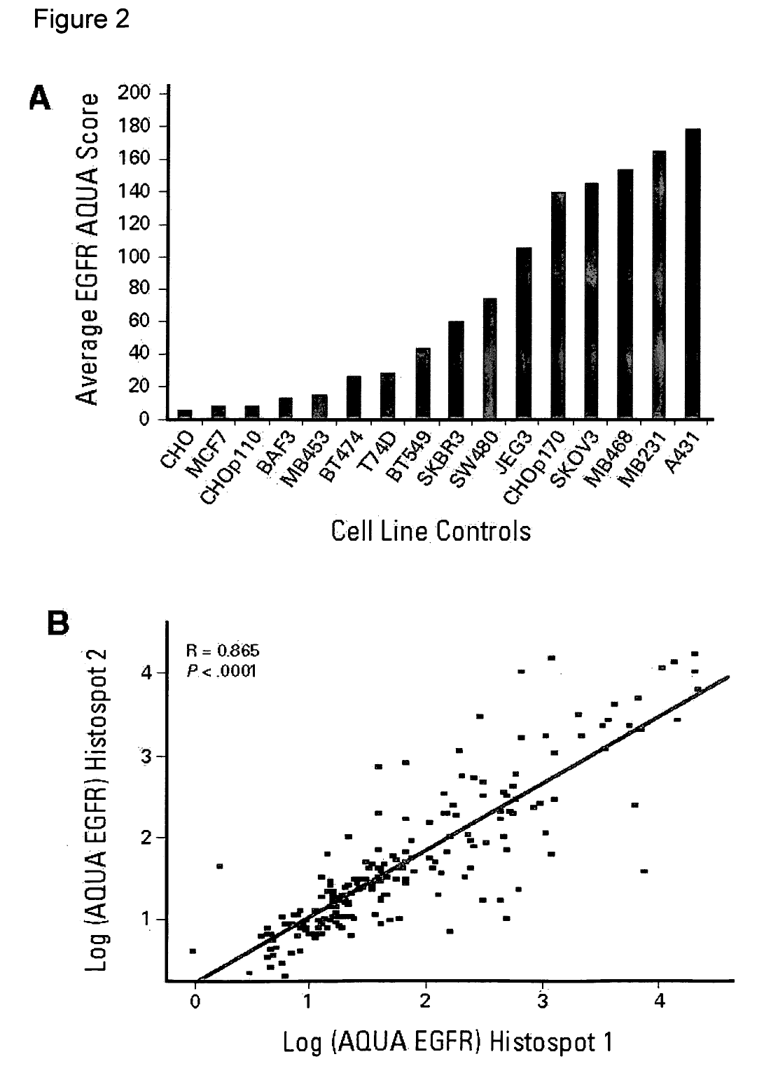

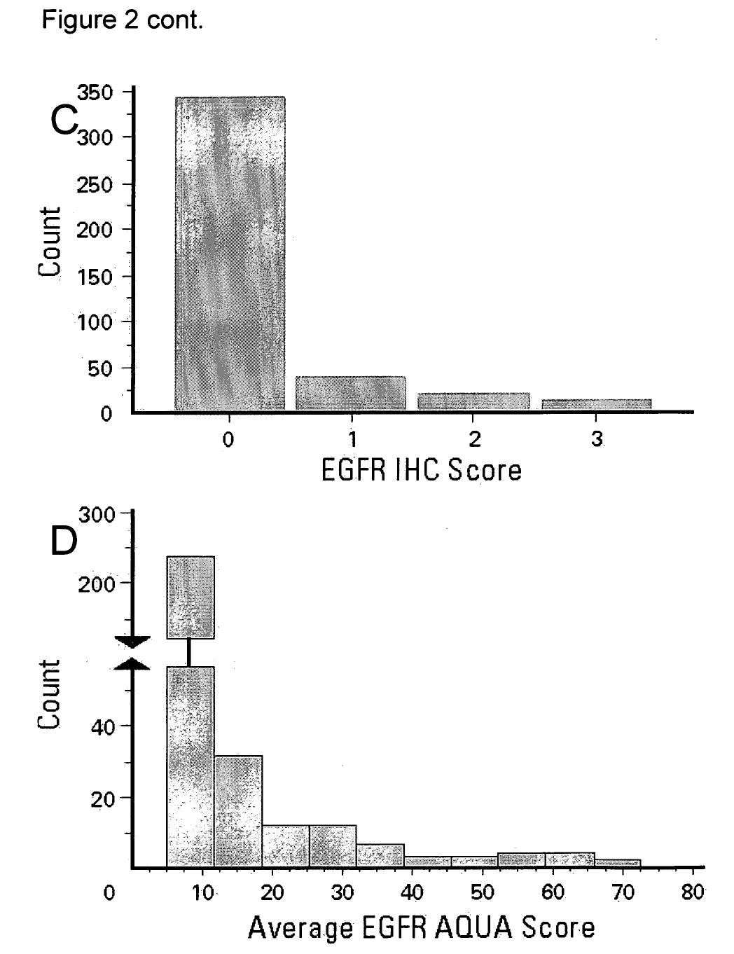

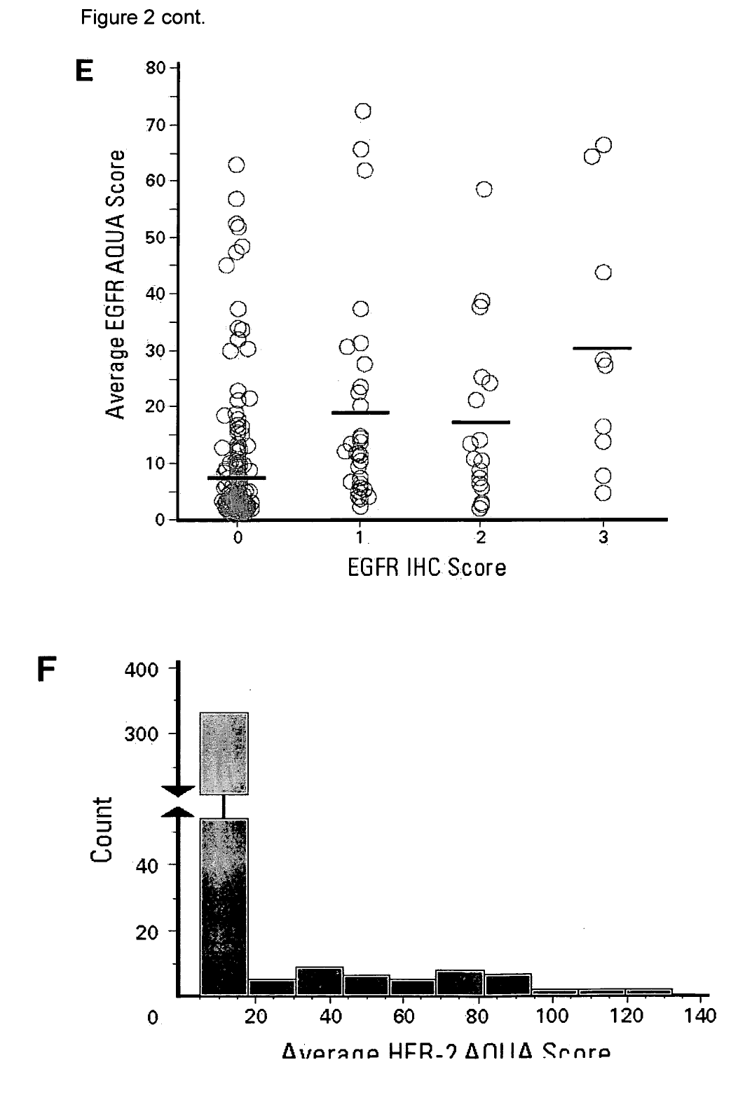

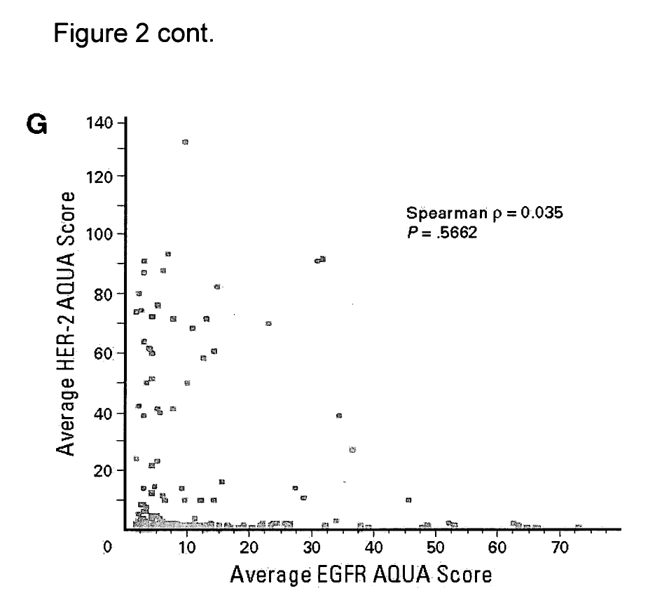

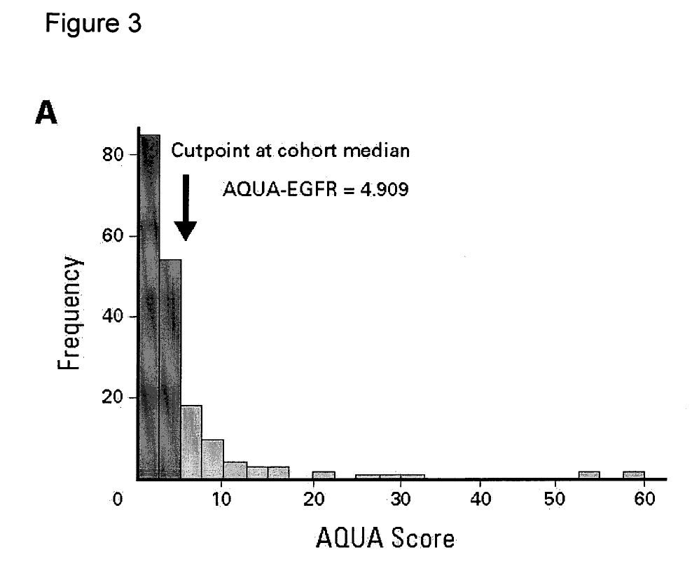

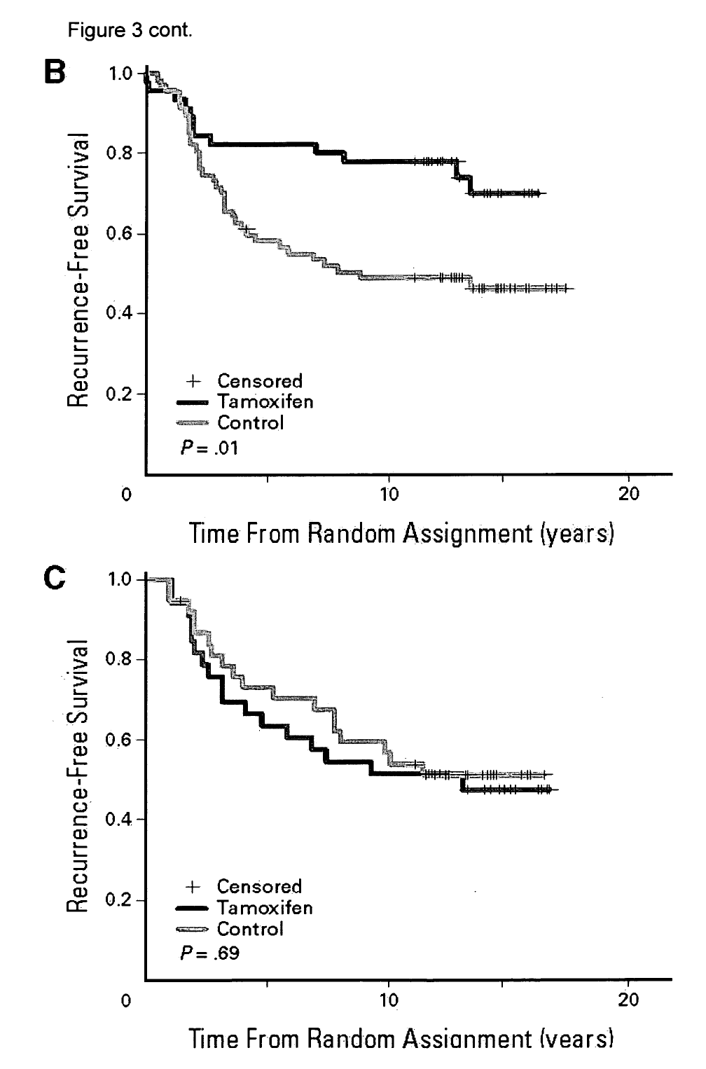

A modified immunofluorescent technique employing the Dako EGFR PharmDx kit was used to stain the SBII:2a tissue microarray. A wide range of staining intensity was observed, and staining was largely confined to the non-nuclear/membranous subcellular compartment, as defined by co-localization with perimembranous cytokeratin expression (FIG. 1). In this cohort, cases with predominant nuclear EGFR staining were not observed.

### Automated Quantitative Analysis (AQUA) of EGFR Expression

Protein expression levels were quantified in the non-nuclear compartment using AQUA software algorithms (FIGS. 1D/H). EGFR expression levels were confirmed using formalin-fixed, paraffin-embedded cell line pellet controls (FIG. 2A). Included in this cell line control series were Chinese hamster ovary cells (CHO), which do not express EGFR, as well as cells stably transfected with soluble (CHO-p110) (22) or full-length (CHO-p170) EGFR (17). Low AQUA scores for CHO and CHO-p110 and a high AQUA score for CHO-p170 cells confirmed that the EGFR PharmDx antibody preferentially bound to full-length, p-170 EGFR (FIG. 2A). In addition, the highest AQUA scores were observed in A431 and MDA-MB-468, cell lines known to carry amplification of the ErbB1/EGFR locus.

In order to assess for intra-tumor heterogeneity of EGFR expression and control for reproducibility of the assay, we compared AQUA scores from redundant tumor cores and observed significant correlation (FIG. 2B, R=0.865, p<0.0001). AQUA scores in the non-nuclear compartment were averaged between the two histospots, and final scores ranging from 1.33-72.523 were obtained for 327 patients (FIG. 2D). A significant number of cases were not interpretable due to insufficient tumor sampling for automated analysis, which requires at least 5% tumor area per histospot. The median AQUA score for the cohort was 4.909, and this was arbitrarily chosen as the cutpoint for classifying tumors as high or low for EGFR expression.

Immunohistochemistry using the EGFR-PharmDx kit was also performed on a separate TMA section, with scores ranging from 0-3 as assessed by a pathologist using Dako scoring guidelines according to the validation schedule used for colon cancer (FIG. 2C). The highest score from two redundant samples was recorded, and positive scores (>0) were observed in 77/402 (19.2%) of patients. We compared quantitative AQUA scores to semi-quantitative IHC scores, and found moderate but significant agreement (FIG. 2E, Spearman Rho=0.426, p<0.0001). Of note, only scores on the high- and low-end of the semi-quantitative scale were easily separated by AQUA scores, an observation that has been made previously (13).¹³ In addition, IHC-based scores of 0 had AQUA scores ranging nearly to the maximum observed in the cohort (range 1.33-63.01). This is likely due to the increased sensitivity of immunofluorescence as well as the calculation method of AQUA scoring, which accounts for both intensity and area of staining (13).

### Survival Analysis and Clinicopathologic Correlations.

AQUA-EGFR scores were not associated with tumor size or presence of lymph node metastasis, but were significantly associated with Nottingham Histological Grade (Spearman Rho=0.241, p<0.0001) . In addition, ER+ patients tended to have lower AQUA-EGFR scores (Spearman Rho−0.402, p<0.0001), confirming previous observations in a similar cohort (2). For this reason, the cutpoint (selected by median AQUA-EGFR value for the entire cohort) , classified 73/186 (39.2%) ER+ patients as EGFR-High (Table 1, FIG. 3A).

We evaluated the association of tamoxifen treatment with RFS in ER+ patients by log rank tests and Kaplan-Meier analysis plots (FIG. 3). In the cohort of patients with low EGFR expression (n=113), there was a significant effect by two years of adjuvant tamoxifen (p=0.01; FIG. 3B), contrasting to no treatment effect in the EGFR high group (n=73) (p=0.69; FIG. 3C). Furthermore, High EGFR expression was associated with a shorter time to recurrence and breast cancer death in tamoxifen-treated patients (Log-Rank p=0.0411), but this was not observed in the control cohort (Log-Rank p=0.466). Tamoxifen treated patients with high EGFR expression had a 52% 10-year RFS, compared to 78% in the EGFR low group.

The relationship between EGFR expression and tamoxifen response in ER+ patients was also explored in a series of Cox proportional hazards models with RFS as endpoint (Table 2). First, the effect of tamoxifen treatment was estimated separately for each of the two EGFR groups, leading to HR=0.43 (95% CI: 0.22-0.84, p =0.013) for patients with low EGFR expression compared to HR=1.14 (95% CI: 0.59-2.22, p=0.7) in the high expression group. To test if these effects are significantly different, a model including EGFR expression (high/low), tamoxifen treatment (+/−) and a treatment-interaction variable (+/−) was fitted. The treatment-interaction variable is significant (p=0.038). When adjusting this model for PR (progesterone receptor) status similar results were achieved, although not strictly significant, with a p-value of 0.10 for the treatment interaction variable.

Finally, in a multivariate Cox proportional hazards model in ER+ patients, Nottingham Histological grade and tumour size were independent prognostic indicators, but tamoxifen treatment and the continuous AQUA-EGFR score were of borderline significance (Table 2B).

Interestingly, analysis of EGFR-scores by the traditional IHC method does not reveal this result. In ER+ cases with low expression of IHC-EGFR (n=232), Tamoxifen treatment had a beneficial effect on RFS (HR 0.57; 95% CI 0.37-0.86), p=0.008), and the treatment effect in the IHC-EGFR-high group (n=27) was non-significant (HR 0.73; 95% CI 0.24-2.28, p=0.6) (Table 2A). Similarly, the interaction between treatment and IHC-EGFR was also not significant (p=0.7), clearly indicating that EGFR scoring by traditional IHC was not able to discriminate between responders and non-responders of adjuvant Tamoxifen treatment.

TABLE 2

| | | | | |
|---|---|---|---|---|
| Cohort | Variable | HR RFS | 95% Confidence Interval | p-value |

(A) Cox univariate proportional hazards

| | | | | | |
|---|---|---|---|---|---|
| Control n = 152 | EGFR-AQUA | 0.99 | 0.61 | 1.50 | 0.5 |
| ER+, | Tamoxifen Treatment | 0.57 | 0.37 | 0.86 | 0.008 |
| EGFR-IHC Low | | | | | |
| n = 232 | | | | | |
| ER+, | Tamoxifen Treatment | 0.73 | 0.24 | 2.28 | 0.6 |
| EGFR-IHC High | | | | | |
| n = 27 | | | | | |
| ER+, | Tamoxifen Treatment | 0.43 | 0.22 | 0.84 | 0.013 |
| EGFR-AQUA Low | | | | | |
| n = 113 | | | | | |
| ER+, | Tamoxifen Treatment | 1.14 | 0.59 | 2.22 | 0.7 |
| EGFR-AQUA High | | | | | |
| n = 73 | | | | | |
| ER+ | Tamoxifen treatment | 0.64 | 0.44 | 0.95 | 0.035 |
| HER2 IHC 0-2+ | | | | | |
| N = 252 | | | | | |
| ER+ | Tamoxifen treatment | 0.67 | 0.21 | 2.22 | 0.5 |
| HER2 IHC 3+ | | | | | |
| N = 23 | | | | | |
| ER+ | Tamoxifen treatment | 0.64 | 0.43 | 0.97 | 0.037 |
| HER2-AQUA Low | | | | | |
| N = 231 | | | | | |
| ER+ | Tamoxifen treatment | 0.28 | 0.05 | 1.41 | 0.12 |
| HER2-AQUA High | | | | | |
| N = 10 | | | | | |

(B) Cox multivariate proportional hazards

| | | | | | |
|---|---|---|---|---|---|
| ER+ | AQUA-HER2 | 1.006 | 0.991 | 1.022 | 0.428 |
| n = 324 | AQUA-EGFR | 1.021 | 1.000 | 1.042 | 0.045 |
| (available data in | Nodal Status (+/−) | 1.563 | 0.827 | 2.954 | 0.169 |
| 186 patients) | NHG (3 v. 1-2) | 2.471 | 1.472 | 4.148 | 0.001 |
| | Tumor Size (T2 v T1) | 2.087 | 1.211 | 3.598 | 0.008 |
| | Age | 0.982 | 0.933 | 1.033 | 0.475 |
| | Tamoxifen Treatment | 0.571 | 0.341 | 0.956 | 0.033 |
| | (+/−) | | | | |

EGFR Expression is not Associated with HER-2/neu Expression

It has been proposed that HER-2/neu expression may influence resistance to tamoxifen treatment, although recent evidence suggests this may be due to the inverse relationship between ER and HER-2/neu and failure to exclude ER− patients from studies of tamoxifen response (2). The results of an AQUA-based analysis of this relationship between EGFR and HER-2/neu expression are shown in FIGS. 2F-G. Although HER2-AQUA scores ranged from 1.14-132.34, only a minority of patients displayed HER-2/neu overexpression (FIG. 2F). EGFR- and HER2-AQUA scores were not correlated in this cohort (FIG. 2G). However, the relationship between HER2/neu analysis by conventional IHC analysis using Herceptest and the AQUA-analysis was significant (Spearman Rho=0.64, p<0.0001) as well as the correlation to ErbB2/HER2 amplification by FISH (Spearman Rho=0.52, p<0.0001).

By using previously defined cutpoints for HER2/neu for the AQUA-based analysis we explored the effect of Tamoxifen treamtent in AQUA-HER2/neu low vs high group of ER+ tumors. A treatment effect was observed in the AQUA-HER2/neu low group (HR 0.64; 95% CI 0.43-0.97, p=0.037), as well as in the AQUA-HER2/neu high group (HR 0.28; 95% CI 0.05-1.41, p=0.12), but the latter was not significant due to low power (n=10). The interaction effect is also insignificant, so no conclusion regarding differential treatment effect in the two AQUA-HER2/neu groups can be made. Results from HER2 analysis in this cohort using Herceptest scoring have previously been published demonstrating no treatment interaction effect and the data from this analysis are provided in Table 2A (16).

### Discussion

Over the last few years, there has been extensive evidence for cross talk between HER family molecules and estrogen receptor.²³ However, there appears to be only a single study that shows clinical evidence of this observation for EGFR (ErbB1/HER1). The cohort described by Arpino and colleagues (3) finds that high levels of both HER1 and HER2 are associated with worse outcome in ER positive patients treated with Tamoxifen (although both are barely significant with p=0.05). In their study, EGFR and HER2 were quantitatively measured by a ligand binding assay in a non-randomized cohort making it difficult to draw any conclusions about the treatment predictive information achieved. Dowsett and colleagues describe two randomized large trials of adjuvant Tamoxifen treatment for two years where the effect of growth factor receptors could be assessed (2). They looked at EGFR and HER2 expression by immunohistochemistry in a fraction of the originally included patients. Unlike the current study, they found EGFR had no relationship with prognosis. Furthermore, there were a very small number of patients that were both ER+ and EGFR positive, so no analysis could be done to assess tamoxifen treatment interaction. However, it is possible that a quantitative assay is needed to detect this effect as proposed by the data shown in this paper where both methods are used to assess treatment interaction. Unfortunately, in this study, many more patient samples were not interpretable by AQUA due to insufficient tumor sampling than “by eye” and this missing data must be considered a limitation of this study.

One possible reason for the limited clinical evidence for the biologically based hypothesis is the difficulty in analysis of EGFR. Detection of EGFR expression by traditional IHC has shown wide variability where some labs show associations and others to do not, even looking at identical questions (24). There are also many antibodies used for the task, some of which may detect isoforms whose function is not well understood (22). Even if all other variables are fixed, there is still the issue of analysis with the human eye contributing to investigator-dependent bias. The cut-point for positive EGFR has been historically considered any positive staining. Similarly, the histogram in FIG. 2 shows the median (chosen as the cut-point in this study) also requires distinction between very low levels of expression (low AQUA scores). A previous study done by our lab analyzing expression of HER2 shows that while there is good correlation between quantitative scores and “by eye” scores at the high end of the scale, that correlation is lost at the low end. In fact by automated analysis, we describe a outcome-based subset of patients that cannot be defined by traditional “by eye” scoring (25).

The options for endocrine therapy treatment in early breast cancer are ever broadening, which has the potential to make this observation more important. For example, a number of studies have show that aromatase inhibitors are superior to Tamoxifen in ER+/PR− patients but similar in ER+/PR+ patients (26,27). Those studies were performed in post-menopausal women where aromatase inhibitors are an option. In this study with pre-menopausal patients, where aromatase inhibitors are not a treatment option, the decision to rely on Tamoxifen alone or in conjunction with chemo-endocrine adjuvant therapies is even more critical. The observations in this paper suggest that careful measurements of EGFR are likely to be valuable in determining pre-menopausal breast cancer patients that are unlikely to respond to Tamoxifen. However, a current cohort of patients treated with only tamoxifen would likely have smaller tumors and less nodal involvement. Future studies should be done in larger, more recent cohorts to test the predictive value of EGFR for finding resistance to both tamoxifen and aromatase inhibitors in both pre- and post-menopausal patients. The recent approvals (in other cancer types) of targeted therapies directed towards the EGFR receptor might suggest a new treatment option in conjunction with tamoxifen.

### References

1. Tamoxifen for early breast cancer: an overview of the randomised trials. Early Breast Cancer Trialists' Collaborative Group. Lancet 351:1451-67, 1998
2. Dowsett M, Houghton J, Iden C, et al: Benefit from adjuvant tamoxifen therapy in primary breast cancer patients according oestrogen receptor, progesterone receptor, EGF receptor and HER2 status. Ann Oncol 17:818-26, 2006
3. Arpino G, Green S J, Allred D C, et al: HER-2 amplification, HER-1 expression, and tamoxifen response in estrogen receptor-positive metastatic breast cancer: a southwest oncology group study. Clin Cancer Res 10:5670-6, 2004
4. Arpino G, Weiss H, Lee A V, et al: Estrogen receptor-positive, progesterone receptor-negative breast cancer: association with growth factor receptor expression and tamoxifen resistance. J Natl Cancer Inst 97:1254-61, 2005
5. Linke S P, Bremer T M, Herold C D, et al: A multimarker model to predict outcome in tamoxifen-treated breast cancer patients. Clin Cancer Res 12:1175-83, 2006
6. Osborne C K, Shou J, Massarweh S, et al: Crosstalk between estrogen receptor and growth factor receptor pathways as a cause for endocrine therapy resistance in breast cancer. Clin Cancer Res 11:865s-70s, 2005
7. Osborne C K: Tamoxifen in the treatment of breast cancer. N Engl J Med 339:1609-18, 1998
8. Johnston S R: Clinical efforts to combine endocrine agents with targeted therapies against epidermal growth factor receptor/human epidermal growth factor receptor 2 and mammalian target of rapamycin in breast cancer. Clin Cancer Res 12:1061s-1068s, 2006
9. Schlessinger J: Cell signaling by receptor tyrosine kinases. Cell 103:211-25, 2000
10. Nicholson R I, McClelland R A, Gee J M, et al: Epidermal growth factor receptor expression in breast cancer: association with response to endocrine therapy. Breast Cancer Res Treat 29:117-25, 1994
11. Nicholson R I, McClelland R A, Finlay P, et al: Relationship between EGF-R, c-erbB-2 protein expression and Ki67 immunostaining in breast cancer and hormone sensitivity. Eur J Cancer 29A:1018-23, 1993
12. Gee J M, Robertson J F, Gutteridge E, et al: Epidermal growth factor receptor/HER2/insulin-like growth factor receptor signalling and oestrogen receptor activity in clinical breast cancer. Endocr Relat Cancer 12 Suppl 1:S99-S111, 2005
13. Camp R L, Chung G G, Rimm D L: Automated subcellular localization and quantification of protein expression in tissue microarrays. Nat Med 8:1323-7, 2002
14. McCabe A, Dolled-Filhart M, Camp R L, et al: Automated quantitative analysis (AQUA) of in situ protein expression, antibody concentration, and prognosis. J Natl Cancer Inst 97:1808-15, 2005
15. Ryden L, Jonsson P E, Chebil G, et al: Two years of adjuvant tamoxifen in premenopausal patients with breast cancer: a randomised, controlled trial with long-term follow-up. Eur J Cancer 41:256-64, 2005
16. Ryden L, Jirstrom K, Bendahl P-O, et al: Tumor-specific expression of vascular endothelial growth factor receptor 2 but not vascular endothelial growth factor or human epidermal growth factor receptor 2 is associated with impaired response to adjuvant tamoxifen in premenopausal breast cancer. J Clin Oncol 23:4695-4704, 2005.
17. Christensen T A, Reiter J L, Baron A T, et al: Generation and characterization of polyclonal antibodies specific for human p110 sEGFR. Hybrid Hybridomics 21:183-9, 2002
18. Dolled-Filhart M, McCabe A, Giltnane J, et al: Quantitative in situ analysis of beta-catenin expression in breast cancer shows decreased expression is associated with poor outcome. Cancer Res 66:5487-94, 2006
19. Dolled-Filhart M, McCabe A, Giltnane J, et al: Quantitative In situ Analysis of {beta}-Catenin Expression in Breast Cancer Shows Decreased Expression Is Associated with Poor Outcome. Cancer Res 66:5487-94, 2006
20. Rubin M A, Zerkowski M P, Camp R L, et al: Quantitative determination of expression of the prostate cancer protein alpha-methylacyl-CoA racemase using automated quantitative analysis (AQUA): a novel paradigm for automated and continuous biomarker measurements. Am J Pathol 164:831-40, 2004
21. Jonat W, Pritchard K I, Sainsbury R, et al: Trends in endocrine therapy and chemotherapy for early breast cancer: a focus on the premenopausal patient. J Cancer Res Clin Oncol 132:275-86, 2006
22. Reiter J L, Maihle N J: Characterization and expression of novel 60-kDa and 110-kDa EGFR isoforms in human placenta. Ann N Y Acad Sci 995:39-47, 2003
23. Schiff R, Massarweh S A, Shou J, et al: Advanced concepts in estrogen receptor biology and breast cancer endocrine resistance: implicated role of growth factor signaling and estrogen receptor coregulators. Cancer Chemother Pharmacol 56 Suppl 1:10-20, 2005
24. Ciardiello F, Tortora G: Epidermal growth factor receptor (EGFR) as a target in cancer therapy: understanding the role of receptor expression and other molecular determinants that could influence the response to anti-EGFR drugs. Eur J Cancer 39:1348-54, 2003
25. Camp R L, Dolled-Filhart M, King B L, et al: Quantitative Analysis of Breast Cancer Tissue Microarrays Shows That Both High and Normal Levels of HER2 Expression Are Associated with Poor Outcome. Cancer Res 63:1445-8, 2003
26. Howell A, Cuzick J, Baum M, et al: Results of the ATAC (Arimidex, Tamoxifen, Alone or in Combination) trial after completion of 5 years' adjuvant treatment for breast cancer. Lancet 365:60-2, 2005
27. Punglia R S, Kuntz K M, Winer E P, et al: The impact of tumor progesterone receptor status on optimal adjuvant endocrine therapy for postmenopausal patients with early-stage breast cancer: a decision analysis. Cancer 106:2576-82, 2006

1. A method for determining the likelihood that a therapy involving administration of tamoxifen to a patient afflicted with a breast cancer will provide a therapeutic benefit to the patient which comprises:
a) determining the level of expression of epidermal growth factor receptor in a breast tissue sample from the patient; and
b) comparing the level of expression so obtained to a predetermined reference level of expression of epidermal growth factor receptor in breast cancer tissue samples of patients who had a lack of therapeutic benefit from such a therapy;
wherein there is a likelihood the therapy will provide the therapeutic benefit to the patient if the level of expression determined in step a) is less than the predetermined reference level of expression.

2. The method of claim 1, wherein the level of expression of epidermal growth factor receptor is determined by measuring the level of epidermal growth factor receptor protein expression.

3. The method of claim 2, wherein the level of epidermal growth factor receptor protein expression is determined by performing an immunohistochemistry procedure.

4. The method of claim 2, wherein the level of epidermal growth factor receptor protein expression is determined by a quantitative image analysis procedure.

5. The method of claim 2, wherein the level of epidermal growth factor receptor protein expression is determined in a cellular compartment within the tissue.

6. The method of claim 2, wherein the level of epidermal growth factor receptor protein expression is determined in a subcellular compartment within the tissue.

7. The method of claim 6, wherein the subcellular compartment is a non-nuclear compartment.

8. The method of claim 6, wherein the subcellular compartment is a cellular membrane compartment.

9. The method of claim 1, wherein the breast cancer is a hormone receptor positive breast cancer.

10. The method of claim 1, wherein the patient is a premenopausal female patient.

11. The method of claim 1, wherein the level of expression of epidermal growth factor receptor is determined using an automated pathology system.

12. The method of claim 1, wherein the therapeutic benefit is progression-free or recurrence-free survival.

13. A method for identifying a patient afflicted with a breast cancer who will likely obtain a therapeutic benefit from tamoxifen therapy which comprises:
a) determining the level of expression of epidermal growth factor receptor in a breast tissue sample from the patient; and
b) comparing the level of expression so obtained to a predetermined reference level of expression of epidermal growth factor receptor in breast cancer tissue samples of patients who had a lack of therapeutic benefit from such a therapy;
wherein the patient will likely obtain the therapeutic benefit from tamoxifen therapy if the level of expression determined in step a) is less than the predetermined reference level of expression.

14. The method of claim 13, wherein the level of expression of epidermal growth factor receptor is determined by measuring the level of epidermal growth factor receptor protein expression.

15. The method of claim 14, wherein the level of epidermal growth factor receptor protein expression is determined by performing an immunohistochemistry procedure.

16. The method of claim 14, wherein the level of epidermal growth factor receptor protein expression is determined by a quantitative image analysis procedure.

17-22. (canceled)

23. The method of claim 13, wherein the level of expression of epidermal growth factor receptor is determined using an automated pathology system.

24. (canceled)

25. A method for determining whether a breast cancer will be responsive to tamoxifen therapy which comprises:
a) determining the level of expression of epidermal growth factor receptor in a breast tissue sample from the patient; and
b) comparing the level of expression so obtained to a predetermined reference level of expression of epidermal growth factor receptor in breast cancer tissue samples of patients who had a lack of therapeutic benefit from such a therapy;
wherein the breast cancer will be responsive to tamoxifen if the level of expression determined in step a) is less than the predetermined reference level of expression.

26-36. (canceled)

37. A method for determining whether a breast cancer will not be responsive to tamoxifen therapy which comprises:
a) determining the level of expression of epidermal growth factor receptor in a breast tissue sample from the patient; and
b) comparing the level of expression so obtained to a predetermined reference level of expression of epidermal growth factor receptor in breast cancer tissue samples of patients who had a lack of therapeutic benefit from such a therapy;
wherein the breast cancer will be not responsive to tamoxifen therapy if the level of expression determined in step a) is greater than the predetermined reference level of expression.

38-48. (canceled)

49. A method for determining the likelihood that a therapy involving administration of an aromatase inhibitor to a patient afflicted with a breast cancer will provide a therapeutic benefit to the patient which comprises:
a) determining the level of expression of epidermal growth factor receptor in a breast tissue sample from the patient; and
b) comparing the level of expression so obtained to a predetermined reference level of expression of epidermal growth factor receptor in breast cancer tissue samples of patients who had a lack of therapeutic benefit from such a therapy;
wherein there is a likelihood the therapy will provide the therapeutic benefit to the patient if the level of expression determined in step a) is less than the predetermined reference level of expression.

50-61. (canceled)
